(19) United States
(12) Patent Application Publication
(10) Pub. No.: US 2025/0268536 A1
(43) Pub. Date: Aug. 28, 2025

(54) Patient Seat and Magnetic Resonance Device

(71) Applicant: Siemens Healthineers AG, Forchheim (DE)

(72) Inventors: Lars Lauer, Neunkirchen (DE); Gunnar Krüger, Binningen (CH); Andreas Greiser, Erlangen (DE); Miriam Keil, Erlangen (DE); Annemarie Hausotte, Erlangen (DE)

(73) Assignee: Siemens Healthineers AG, Forchheim (DE)

(21) Appl. No.: 19/058,126

(22) Filed: Feb. 20, 2025

(30) Foreign Application Priority Data
Feb. 22, 2024 (EP) 24159090.0

(51) Int. Cl. A61B 5/00; A61B 5/055; A61B 90/00

(52) U.S. Cl. CPC A61B 5/70; A61B 5/055; A61B 90/03

(57) ABSTRACT

A patient seat for supporting a patient during a magnetic resonance examination, having a first portion, a second portion, a connecting element, a radio-frequency unit with at least one antenna element, and a drive unit, wherein the first portion and the second portion form parts of a receiving surface for the patient, wherein the connecting element mechanically connects the first portion to the second portion and is configured to enable a variable relative movement between the first portion and the second portion, wherein the at least one antenna element of the radio-frequency unit is configured to receive signals in a power and frequency range of a magnetic resonance examination, and wherein the drive unit is configured to move the patient seat variably along a spatial direction.

### BACKGROUND

Independent of the grammatical term usage, individuals with male, female, or other gender identities are included within the term.

For the use of dedicated magnetic resonance devices, in particular dedicated head scanners or dedicated dental scanners, in smaller medical facilities and practices, the smallest possible space requirement, the simplest possible and most time-efficient patient positioning, and a high level of patient comfort are of crucial importance. A reduction in the size of magnetic resonance devices is usually accompanied by a reduction in the imaging volume, which increases demands on the accuracy of patient positioning. In addition, accurate patient positioning requires trained staff, which can, in particular, be a problem for smaller medical facilities, both logistically and financially.

Nowadays, with very few exceptions, magnetic resonance devices use horizontally aligned patient tables with a substantially planar lying surface. With conventional patient tables of this type, a receiving coil is first manually arranged on a diagnostically relevant body region of a patient in a two-stage workflow before a center offset of the receiving coil from a given reference point is determined for subsequent iso-centering by moving the patient table along an access direction of the magnetic resonance device (typically a longitudinal axis of a patient receiving region or a Z direction of the magnetic resonance device). Conventional patient tables have been found to be unsuitable, especially in smaller medical facilities and practices, because they require a relatively large amount of space and involve intricate workflows. Furthermore, to date, “precise” iso-centering has only been possible parallel to the longitudinal axis of the patient table but not along other spatial directions. Known patient seats, such as those used in some extremity scanners, reduce the space required, but they do not reduce the workload associated with patient positioning.

### SUMMARY

It is, therefore, an object of the disclosure to provide a patient seat and a magnetic resonance device with smaller dimensions than conventional systems and to enable the simplification of a patient positioning workflow.

According to the disclosure, this object is achieved by the subject matter of the independent claims. Advantageous aspects and expedient developments are the subject matter of the subclaims.

The patient seat according to the disclosure is embodied to support a patient during a magnetic resonance examination. The patient seat is preferably embodied to support and/or hold in a predetermined spatial position a patient positioned on the patient seat appropriately for the application, in particular, a diagnostically relevant body region of the patient. It is likewise conceivable that the patient seat is embodied to maintain a predetermined posture of the patient at least for the duration of the magnetic resonance examination.

The patient seat has a first portion, a second portion, a connecting element, a radio-frequency unit with at least one antenna element, and a drive unit.

The first portion and the second portion can be any part of the patient seat. The first portion is preferably designed as a seat surface of the patient seat. The second portion can be designed as a backrest of the patient seat. It is furthermore conceivable that the first portion or the second portion is designed as a footrest or a headrest. Furthermore, a portion of the patient seat designed as a backrest can comprise a headrest. The headrest can be arranged in a fixed arrangement relative to the backrest or integrated into the backrest. However, it is likewise also conceivable that the headrest is connected to a portion of the patient seat by means of the connecting element, a further connecting element or a positioning unit. The headrest can, in particular, be a portion, for example, a third portion or a fourth portion, of the patient seat.

According to the disclosure, the first portion and the second portion form parts of a receiving surface for the patient. This can mean that the first portion and the second portion are embodied to hold or support selected or assigned body regions of a patient positioned on the patient seat appropriately for the application. Herein, the first portion and the second portion are preferably in direct contact with the selected or assigned body regions of the patient.

The connecting element mechanically connects the first portion to the second portion and is embodied to enable variable relative movement between the first portion and the second portion. For example, the connecting element is embodied to align the backrest of the patient seat variably relative to the seat surface of the patient seat.

In a preferred aspect, the first portion and the second portion can be arranged at an angle to one another by means of the connecting element, in particular at an angle different from 0° or 180°.

The connecting element can comprise a joint, a guide element, a gear, and/or an elastic element or be designed as a joint, a guide element, a gear, and/or an elastic element. For example, the connecting element can comprise a mechanical spring, an angle joint, a ball joint, a radial bearing, a thrust bearing, a coupling gear, a straight guide, an axle, and/or a shaft.

The connecting element is preferably embodied to enable positioning of the second portion relative to the first portion as a result of manual actuation by a user (for example, the patient or a member of the medical staff) and/or actuation by a positioning unit according to an aspect described below.

It is furthermore conceivable that the patient seat comprises more than two portions, which are embodied to be moved variably relative to one another. The connecting element is preferably embodied to move the portions of the patient seat, such as, for example, a seat surface, a backrest, a headrest and/or a footrest, variably relative to one another. However, it is likewise conceivable that the patient seat has a plurality of connecting elements, which are embodied to move the portions of the patient seat variably relative to one another. For example, the patient seat can have a further connecting element, which is embodied to move a headrest variably relative to a backrest.

The at least one antenna element of the radio-frequency unit is embodied to receive signals in a power and frequency range of a magnetic resonance examination.

The at least one antenna element preferably has one or more signal conductors. In particular, the at least one antenna element can be a coupling element between electromagnetic waves guided in signal conductors and unguided electromagnetic waves, i.e., electromagnetic waves in free space. The at least one antenna element is preferably embodied to receive electromagnetic waves in the range of the magnetic resonance frequency of a magnetic resonance-active atomic nucleus. Herein, by way of example, an electromagnetic wave with a frequency of between 1 and 500 MHZ, preferably between 10 and 300 MHz, is considered to be a radio-frequency signal. The magnetic resonance signal of the usual atomic nuclei to be examined can have a low power of a few microwatts to several milliwatts.

A signal conductor preferably comprises an electrically conductive wire. The signal conductor wire is preferably embodied to transmit the aforementioned powers continuously. For example, the signal conductor can be embodied as a free wire, as a wound coil, or as a conductor track on a printed circuit board of the at least one antenna element. The signal conductor is preferably made of copper. However, other electrically conductive metals, such as, for example, gold, silver or aluminum, are also conceivable.

The at least one antenna element can have one or more coil-shaped signal conductors. It is conceivable that the at least one antenna element comprises a butterfly coil that can be folded or pivoted along a patient's anatomy, in particular the temporomandibular joint region.

In a preferred aspect of the patient seat, the radio-frequency unit is arranged on a head region of the patient when the patient is positioned on the patient seat appropriately for the application. The radio-frequency unit can be designed as a head coil, in particular as a dental coil. The at least one antenna unit of the radio-frequency unit can be designed as a receiving coil and/or a transmitting coil. It is conceivable that the radio-frequency unit comprises a plurality of antenna elements, in particular, at least one receiving coil and at least one transmitting coil.

In a particularly preferred aspect, the radio-frequency unit is permanently or irreversibly integrated into a portion of the patient seat, in particular, the headrest or the backrest. It is conceivable that the radio-frequency unit of the patient seat forms part of a radio-frequency system of a magnetic resonance device and/or is designed to be actuatable by a radio-frequency control unit of a magnetic resonance device.

The drive unit is embodied to move the patient seat variably along a spatial direction. The drive unit is preferably embodied to move the patient seat along a patient access direction of a magnetic resonance device. The patient access direction can coincide with a longitudinal direction of a patient receiving region of the magnetic resonance device (for example, a Z direction) or be aligned parallel to the longitudinal direction of a patient receiving region of the magnetic resonance device. However, the spatial direction, and also the patient access direction of the magnetic resonance device, can deviate from the horizontal or be aligned at an angle different from zero degrees (and/or 180 degrees), for example, an angle between 1 and 70°, preferably an angle between 5 and 45°, to the horizontal.

The drive unit can comprise any kind of drive, for example, an electric or pneumatic drive, in particular, a hydraulic drive. It is furthermore conceivable that the drive unit comprises a guide element, such as, for example, a gear, a bearing, a rail, a guide rod, an axle, a shaft, and/or a linear guide, which is embodied to mechanically couple the patient seat to the drive unit. The drive unit can accordingly be embodied to move the patient seat along a movement trajectory specified by the guide element and/or the drive unit.

The patient seat according to the disclosure advantageously enables dimensions to be reduced compared to conventional patient support apparatuses for use in magnetic resonance imaging. For example, the patient seat according to the disclosure can enable a magnetic resonance examination on a patient sitting upright or a patient leaning back with a dedicated magnetic resonance device, whereby location requirements, such as, for example, the size of an examination room, the load-bearing capacity of a floor and/or the dimensions of an access route to the examination room, can advantageously be mitigated. Furthermore, the division of the patient seat into at least the first portion and the second portion enables an angled arrangement of body regions or extremities relative to an upper body of the patient. This enables the patient's muscles, joints, and spine to be advantageously relieved of pressure, whereby the patient's comfort during the magnetic resonance examination can be increased, and/or the risk of the magnetic resonance examination being aborted can be reduced.

A radio-frequency unit integrated into the patient seat makes it possible to avoid work steps associated with the arrangement or attachment of the radio-frequency unit, whereby the efficiency of patient preparation for the magnetic resonance examination is advantageously increased. Furthermore, the integration of the radio-frequency unit into the patient seat enables loose electrical cables and/or other parts and components of the radio-frequency unit in the vicinity of the patient to be avoided, whereby irritation of the patient and also wear of mechanical and electrical parts of the radio-frequency unit due to contact with the patient can be advantageously avoided or reduced.

The patient seat according to the disclosure can be embodied to receive the patient in an upright sitting posture. It is conceivable that the patient's posture can be adjusted manually, automatically, or partially automatically by means of the connecting element and/or a positioning unit in order to transfer the patient from an upright sitting posture into a posture suitable for the magnetic resonance examination.

A patient seat according to the disclosure enables independent positioning of the patient without further adaptation, correction, or assistance from medical staff. In particular, a patient seat with two portions aligned at an angle to one another can define application-appropriate positioning of the patient. Further adaptation of the patient's posture for performing the magnetic resonance examination can be carried out by means of a relative arrangement of the first portion and the second portion, whereby time-consuming patient positioning is advantageously avoided.

In a preferred aspect, the patient seat according to the disclosure or its components, such as for example, the first portion, the second portion, the connecting element, a positioning unit, and/or the drive unit, are made of a material that is compatible with a magnetic resonance examination. This can mean that the material avoids interference with the magnetic resonance examination, in particular, the occurrence of image artifacts.

In one aspect, the patient seat according to the disclosure has a positioning unit, which is embodied to automatically position the first portion variably relative to the second portion.

The positioning unit can be part of the connecting element or comprise the connecting element. It is conceivable that the positioning unit has a drive, which is embodied to position the first portion variably relative to the second portion. The drive of the positioning unit can be designed as an electric drive, a pneumatic drive, or a hydraulic drive. It is furthermore conceivable that the positioning unit is embodied to store energy provided by the drive. For example, the positioning unit can comprise a mechanical spring or a gas pressure spring, which is embodied to store energy provided by the drive and convert it in a controlled manner into a change in the arrangement of the first portion relative to the second portion.

Preferably, the connecting element and/or the positioning unit have at least one securing element, which is embodied to limit a movement of the first portion relative to the second portion. For example, the securing element can be designed as a stop element. A securing element can be embodied to define or limit a range of movement between the first portion and the second portion. It is, in particular, conceivable that a guide element of the connecting element and/or the positioning unit has a securing element that restricts an angle of rotation and/or an angle of tilt of the second portion relative to the first portion.

A positioning unit according to the disclosure enables a posture of a patient arranged on the patient seat to be advantageously adapted in a time-efficient manner in dependence on a body region of interest.

In one aspect of the patient seat according to the disclosure, the drive unit is embodied to position the patient seat variably along a first spatial direction and a second spatial direction, which is aligned orthogonally to the first spatial direction.

The first spatial direction can, for example, coincide with a patient access direction and/or a Z direction of a magnetic resonance device. In contrast, the second spatial direction can coincide with a Y direction of a magnetic resonance device and/or an anterior-posterior direction of a patient's upper body positioned on the patient seat appropriately for the application.

In one aspect, the drive unit is embodied to position the patient seat variably along a third spatial direction, which is aligned orthogonally to the first spatial direction and the second spatial direction.

In a preferred aspect, the first spatial direction deviates from an imaginary horizontal. For example, a longitudinal axis of a patient receiving region of the magnetic resonance device can be inclined relative to the imaginary horizontal, so that the Z direction or the patient access direction of the magnetic resonance device are also angled or inclined relative to the imaginary horizontal. Hence, the drive unit can, in particular, be embodied to move the patient seat variably along a trajectory that has an angle different from zero to the imaginary horizontal.

The patient seat according to the disclosure enables a spatial position of a diagnostically relevant body region of a patient positioned on the patient seat appropriately for the application to be automatically coordinated along several spatial directions with a spatial position of an imaging volume of a magnetic resonance device. This can reduce or completely eliminate the need for medical staff to assist with patient positioning. Furthermore, diagnostically relevant body regions can advantageously be positioned with high accuracy by means of the patient seat according to the disclosure, whereby magnetic resonance devices with small imaging volumes and a small footprint can also be used.

An imaging volume can be characterized by a predetermined magnetic field direction and/or a predetermined magnetic field strength. For example, the imaging volume can comprise a volume with a substantially uniform magnetic field direction and/or a substantially homogeneous magnetic field strength. An imaging volume can coincide with an iso-center of a magnetic resonance device.

Furthermore, an inclined patient access direction and a drive unit, which is embodied to move the patient seat at an angle to an imaginary horizontal, advantageously enable the footprint of the patient seat and/or of the magnetic resonance device to be reduced.

In a preferred aspect, the patient seat according to the disclosure has a headrest and a further connecting element.

The headrest can be mechanically coupled or connected to the first portion and/or the second portion of the patient seat by means of the further connecting element.

It is conceivable that the further connecting element is designed as part of a positioning unit or a further positioning unit according to an aspect described herein. The further connecting element can also comprise the further positioning unit.

The further connecting element can be designed in accordance with an aspect of the connecting element. According to the disclosure, the further connecting element is embodied to position the headrest variably relative to the first portion and/or the second portion, wherein the radio-frequency unit is arranged on the headrest.

The headrest can be embodied to support or stabilize the head or a spatial position of the head of a patient positioned on the patient seat appropriately for the application.

The further connecting element is preferably embodied to automatically change a spatial position of the headrest relative to a spatial position of the second portion and/or the first portion. For example, the further connecting element is embodied to move the headrest relative to a backrest of the patient seat.

A headrest according to the disclosure advantageously enables a spatial position and/or alignment of the head of a patient positioned on the patient seat appropriately for the application to be adapted. As a result, a patient's head can be spatially positioned and/or aligned independently of other regions of the patient's body in order to coordinate a spatial position of a diagnostically relevant region of the head with a spatial position of an imaging volume of a magnetic resonance device. In contrast to conventional patient support apparatuses, this advantageously enables time-consuming positioning of the patient's entire body to be avoided.

Furthermore, automatic positioning of the patient's head by means of the headrest of the patient seat according to the disclosure enables more precise spatial positioning of a diagnostically relevant body region compared to manual positioning. As a result, it is advantageously possible to use magnetic resonance devices with a lower magnetic field strength and/or smaller imaging volumes that require less space.

In one aspect, the radio-frequency unit is arranged on the headrest of the patient seat. For example, the radio-frequency unit is mechanically connected to the headrest or integrated into the headrest.

Arranging the radio-frequency unit on the headrest enables the at least one antenna element to be held by the headrest and spatially positioned and/or aligned together with the headrest by means of a connecting element and/or a positioning unit according to an aspect described herein. As a result, it is advantageously possible to avoid a separate mechanism for adjusting a relative position between the radio-frequency unit and the patient seat.

In particular, the arrangement of the radio-frequency unit on the headrest makes it possible to maintain a predetermined position between the head of a patient positioned on the patient seat appropriately for the application and the at least one antenna unit. As a result, it is advantageously possible to avoid errors when manually positioning the radio-frequency unit on a diagnostically relevant region of the patient.

In a further aspect of the patient seat according to the disclosure, the further connecting element is embodied to enable variable positioning of the headrest substantially parallel to an anterior-posterior direction and/or a superior-inferior direction of a patient's upper body positioned on the patient seat appropriately for the application.

An anterior-posterior direction of a patient's upper body positioned on the patient seat appropriately for the application can coincide with a Y direction of a magnetic resonance device used for the magnetic resonance examination. In contrast, a superior-inferior direction of a patient's upper body positioned on the patient seat appropriately for the application can coincide with a Z direction of a magnetic resonance device used for the magnetic resonance examination. It is conceivable that the anterior-posterior direction of the patient's upper body deviates by an angle of up to 15°, up to 30°, or up to 45° from the Y direction of the magnetic resonance device used. Likewise, the superior-inferior direction of the patient's upper body can deviate by an angle of up to 15°, up to 30°, or up to 45° from the Z direction of the magnetic resonance device used.

In one aspect, the further connecting element is embodied to rotate or pivot the headrest about an axis aligned substantially parallel to a medial direction of the patient when the patient is positioned on the patient seat appropriately for the application.

Variable positioning of the headrest enables particularly time-efficient coordination of a spatial position of a large number of diagnostically relevant regions of the head with a spatial position of an imaging volume of a magnetic resonance device with little technical effort.

In one aspect of the patient seat according to the disclosure, the connecting element is embodied to move the first portion variably relative to the second portion such that a relative position of the head of a patient positioned on the patient seat appropriately for the application and a portion of the patient seat remains substantially unchanged.

The connecting element can, in particular, be embodied to move the first portion variably relative to the second portion such that a relative position of the head of a patient positioned on the patient seat appropriately for the application and a portion of the patient seat supporting the patient's head, for example, a headrest and/or a backrest, remains substantially unchanged.

It is conceivable that the connecting element and/or the positioning unit are embodied to change a relative spatial arrangement of the first portion and the second portion in dependence on an anatomically correct movement pattern, in particular, a model of a person. For example, the connecting element can be embodied to move a backrest relative to a seat surface of the patient seat such that a relative position between a back portion of a patient who is leaning back on the patient seat appropriately for the application and the backrest remains substantially unchanged.

Furthermore, the connecting element and/or the further connecting element can be embodied to move a headrest relative to a seat surface of the patient seat such that a relative position between the head of the patient who is leaning back on the patient seat appropriately for the application and the headrest remains substantially unchanged.

The connecting element and/or the further connecting element can have a coupling gear or a plurality of guide elements, for example, a joint and a plain bearing, a plurality of joints, or one or more joints and bearings. The coupling gear or the plurality of guide elements can be embodied to move a portion of the patient seat along an elliptical movement trajectory relative to a further portion of the patient seat. For example, the connecting element and/or the further connecting element can be embodied to shift the backrest in a vertical direction relative to the seat surface during rotation. This enables a relative movement between the backrest and a back portion of the patient, but also the head and the headrest, to be avoided.

It is likewise conceivable that the positioning unit and/or the further positioning unit are embodied to change a relative spatial arrangement of the first portion and the second portion, but also a relative spatial arrangement of a third portion and the second portion, by means of the connecting element and/or the further connecting element in dependence on an anatomically correct movement pattern or model of a person. Such a movement pattern or model of a person can, for example, be read from a database and processed by means of a computing unit. A control unit can be embodied to output a control command, which actuates the positioning unit and/or the further positioning unit based on the movement pattern or model of the person. The computing unit and the control unit can be integrated into the patient seat or a magnetic resonance device or designed as stand-alone components.

A patient seat according to the disclosure enables patients to position themselves independently on the patient seat by assuming a sitting position. Avoiding a relative movement between portions of the patient seat and body regions of the patient corresponding to the portions enables guidance of the patient by members of the medical staff to be avoided and the amount of work involved in patient positioning to be reduced.

Furthermore, parts of a radio-frequency unit, in particular the at least one antenna element, can already be arranged on a patient positioned upright in the patient seat. As a result, the process of arranging a part of a radio-frequency unit relative to the patient can advantageously be simplified. In particular, a member of the medical staff can adopt a more ergonomic posture when the at least one antenna element is arranged on a patient sitting upright compared to a patient lying down, whereby the occurrence of posture damage or posture-related clinical pictures can be avoided or reduced.

In a further aspect, the radio-frequency unit of the patient seat according to the disclosure has a guide element, which is embodied to move at least one antenna element variably relative to a portion of the patient seat.

A guide element can be designed according to an above-described aspect. In particular, the guide element can comprise pivot bearings, a plain bearing, a joint, a hinge and/or a folding element. The guide element is preferably embodied to move or rotate at least a portion of the at least one antenna element about an axis point defined by the guide element. The guide element can be arranged at an end of the at least one antenna element or divide the at least one antenna element into a plurality of portions.

It is conceivable that the radio-frequency unit has one or more joints, which are embodied to move a plurality of portions of the at least one antenna element variably relative to one another. The one or more joints can furthermore be embodied to move the at least one antenna element or the plurality of portions of the at least one antenna element variably relative to the radio-frequency unit and/or the second portion of the patient seat.

In a preferred aspect, the guide element is designed as a thrust bearing or a hinge, which is embodied to pivot or rotate the at least one antenna element about an axis aligned parallel to a sagittal plane of an upper body, in particular the head, of a patient positioned on the patient seat appropriately for the application.

In a further aspect, the radio-frequency unit has at least one second antenna element and a further guide element according to an above-described aspect. The at least one antenna element and the second antenna element are preferably arranged or attached to the patient seat such that they flank a patient positioned on the patient seat appropriately for the application from two opposite sides.

In one aspect, the guide element is embodied to move the at least one antenna element in discrete steps or distances relative to a portion of the patient seat. For this purpose, the guide element can, for example, have a latching mechanism and/or a latching element.

A radio-frequency unit according to the disclosure with a guide element allows a particularly time-efficient arrangement of the at least one antenna element on a patient arranged on the patient seat. In particular, an arrangement of the at least one antenna element relative to the patient in discrete steps or distances enables efficient adaptation to a size of a body region of a patient. Furthermore, an antenna element with a guide mechanism according to the disclosure enables the provision of an improved or optimized signal-to-noise ratio.

In a further aspect of the patient seat according to the disclosure, the radio-frequency unit has a pivoting mechanism, which is embodied to pivot the at least one antenna element about an axis aligned substantially parallel to a medial direction of the patient when the patient is positioned on the patient seat appropriately for the application.

A pivoting mechanism can comprise a guide element according to an above-described aspect. The pivoting mechanism preferably has at least one pivot bearing, in particular a thrust bearing, or a hinge.

The pivoting mechanism can define a pivot axis or an axis of rotation, which is aligned substantially parallel to a medial direction or a lateral direction, in particular a transverse plane, of the upper body or the head of a patient positioned on the patient seat appropriately for the application.

It is conceivable that the pivoting mechanism is embodied to pivot the at least one antenna element on one side of the patient about said axis of rotation. The radio-frequency unit preferably has at least one further antenna element. The at least one antenna element and the further antenna element can be held on the patient seat by means of the pivoting mechanism such that they flank a patient positioned on the patient seat appropriately for the application from two opposite sides. It is furthermore conceivable that the at least one antenna element and the further antenna element are coupled by means of the pivoting mechanism and can be pivoted synchronously with one another about the pivot axis of the pivoting mechanism.

A radio-frequency unit according to the disclosure with a pivoting mechanism enables a plurality of antenna elements to be arranged in a predetermined position relative to the patient from one side of the patient seat. As a result, it is advantageously possible to avoid a work step of separate positioning of antenna elements.

Furthermore, antenna elements attached to the patient seat enable a reduction in at least one degree of freedom of movement compared to conventional support coils and removable local coils, whereby a work step of positioning and/or aligning the at least one antenna element can be performed with reduced time expenditure.

The aspects of the radio-frequency unit described herein can enable the at least one antenna element to be arranged on a patient without special expertise, whereby time-consuming training of medical staff can advantageously be avoided.

The magnetic resonance device according to the disclosure is suitable for performing a magnetic resonance examination on a patient arranged in a patient receiving region of the magnetic resonance device.

The magnetic resonance device is preferably embodied to perform a magnetic resonance examination on a patient positioned within an image recording region of the magnetic resonance device, in particular on a patient arranged on a patient seat according to the disclosure appropriately for the application. The image recording region can substantially coincide with the patient receiving region or form a part of the patient receiving region. The magnetic resonance device is preferably embodied to capture magnetic resonance data or magnetic resonance signals from the patient. Furthermore, the magnetic resonance device can be embodied to capture magnetic resonance image data, in particular diagnostic magnetic resonance image data, from the patient positioned within the image recording region.

The magnetic resonance device can comprise a gradient system with one or more gradient coils. Furthermore, the magnetic resonance device can comprise a radio-frequency coil, in particular, a body coil permanently integrated into the magnetic resonance device. In a preferred aspect, the gradient coil(s) and the radio-frequency coil have electrical conductor structures, which are shell-shaped or cylindrical and enclose the image recording region of the magnetic resonance device along a patient access direction. It is conceivable that the gradient coil(s) enclose the radio-frequency coil along the patient access direction.

In a preferred aspect, the patient access direction of the magnetic resonance device is arranged at an angle other than zero degrees to an imaginary horizontal. It is conceivable that a longitudinal axis defined by the patient receiving region of the magnetic resonance device is inclined in the direction of a substantially horizontal floor surface of an examination room in which the magnetic resonance device is installed. For example, an angle between an orthogonal to the floor surface and the patient access direction can be less than 90°, preferably less than 80°, particularly preferably less than 70°.

In a preferred aspect, the magnetic resonance device according to the disclosure is designed as a closed-bore scanner or a scanner with a cylindrically shaped image recording region. A closed-bore scanner can have a substantially cylindrically shaped image recording region. A main magnet of the closed-bore scanner can comprise one or more solenoid coils enclosing the image recording region along an axial direction or an axis of rotation, in particular, an axis of rotational symmetry, of the main magnet. A solenoid coil can comprise an electrical conductor with negligible electrical resistance at (or below) a superconducting temperature. A direction of a main magnetic field provided by the main magnet can be aligned substantially parallel to the patient access direction and/or the axial direction of the patient receiving region.

It is likewise conceivable that the magnetic resonance device according to the disclosure is designed as an open-bore scanner. A main magnet of an open-bore scanner can comprise two magnets, which are separated from one another by the image recording region. A direction of the main magnetic field of the open-bore scanner can be aligned substantially orthogonal to a patient access direction to the image recording region and/or a longitudinal direction of the image recording region.

The main magnet of the magnetic resonance device can comprise or consist of one or more electromagnets or superconducting magnets. In a preferred aspect, the main magnet comprises or consists of one or more cylindrically shaped superconducting magnets or superconducting coils. The main magnet can be mechanically coupled to a magnetic holding structure and/or attached to the magnetic holding structure. The magnetic holding structure is preferably embodied to carry and/or support the main magnet. The term “main magnet” can comprise one or more magnets or coils and a dedicated support structure for the magnets or coils.

In a preferred aspect, the magnetic resonance device according to the disclosure is a “dry” system. A “dry” system can contain a small amount of cryogen or no cryogen at all. For example, the magnetic resonance device according to the disclosure can comprise one or more cryogen containers, which are thermally connected to the main magnet by means of a heat conducting structure. A cryogen container of a “dry” system can contain a volume of less than 10 liters, less than 5 liters, or less than 1 liter of cryogen. In one aspect, cryogen containers are not used. In this case, the main magnet is completely cooled by means of a heat conducting structure. The use of a “dry” system enables the weight of the magnetic resonance device to be reduced, but also enables an infrastructure (for example a so-called quench pipe) associated with the escape of cryogen in the event of a quench to be avoided. As a result, it is advantageous to reduce the space requirement and also further location requirements of the magnetic resonance device according to the disclosure.

In an alternative aspect, the magnetic resonance device is designed as a “wet” system. A “wet” system can comprise at least one cryogen container with a volume of more than 10 liters. In “wet” systems, the main magnet is preferably arranged within the cryogen container and cooled directly by the cryogen.

A cryogen can be a fluid with a low boiling point, such as, for example, argon, nitrogen, neon, helium or the like. A cooling temperature of the cryogen can substantially correspond to the superconducting temperature of the main magnet.

The concepts according to the disclosure described herein can also be transferred to magnetic resonance devices with main magnets comprising or consisting of permanent magnets.

The magnetic resonance device has a patient seat according to an above-described aspect.

According to the disclosure, the drive unit is embodied to move the patient seat variably relative to the patient receiving region of the magnetic resonance device along at least one spatial direction.

The drive unit is preferably embodied to position the patient seat variably along a Z direction and/or a patient access direction. The drive unit can, in particular, be embodied to transport the at least one part of the patient seat and a patient positioned on the patient seat appropriately for the application along the patient access direction into the image recording region of the magnetic resonance device. The spatial direction can, however, also be inclined relative to the patient access direction or at an angle to the patient access direction.

Furthermore, the drive unit can be embodied to transport the patient seat variably along a second and/or a third direction, which are aligned orthogonally to the spatial direction. The drive unit is preferably embodied to move the patient seat variably along the spatial direction, the second spatial direction, and/or the third spatial direction.

The longitudinal axis of the patient receiving region of the magnetic resonance device is preferably inclined relative to an imaginary horizontal and coordinated with the spatial direction, along which the drive unit can transport the patient seat such that it is possible to reduce the space requirement compared to conventional magnetic resonance apparatuses in which the patient is transported along a horizontal by means of a patient support apparatus.

The magnetic resonance device according to the disclosure shares the advantages of the patient seat according to the disclosure.

In particular, the magnetic resonance device according to the disclosure and the patient seat according to the disclosure can advantageously provide a reduction in the space required for a magnetic resonance examination and reduce the workload associated with patient positioning.

In one aspect, the magnetic resonance device according to the disclosure has a securing element, which is embodied to limit an extent of the alignment of the first portion relative to the second portion in order to avoid a collision of the patient seat and/or of a patient positioned on the patient seat appropriately for the application with a housing portion of the magnetic resonance device.

The securing element can, for example, comprise a pin, a bolt, a latching mechanism, a screw, a stop element, a damping element, or the like. The securing element can be embodied to interact or mechanically engage with the connecting element and/or the positioning unit of the patient seat. It is likewise conceivable that the securing element forms part of the connecting element and/or the positioning unit.

In one aspect, the securing element is embodied to limit a movement of the connecting element and/or the positioning unit. The securing element can be embodied to mechanically engage with the connecting element and/or the positioning unit. The securing element can be embodied to permanently limit a range of movement between the first portion and the second portion. It is, however, likewise conceivable that the securing element is embodied to be transferred in dependence on a control command to a securing position in which the securing element limits the relative alignment or movement of the first portion relative to the second portion.

The securing element can have a suitable drive, which can be actuated by means of a control unit of the magnetic resonance device and/or the patient seat. The drive of the securing element can be embodied to activate the securing element or to limit the extent of the relative alignment of the first portion to the second portion by activating the securing element. In particular, the drive can have a signal connection with a stand-alone control unit or a control unit integrated into the magnetic resonance device. The drive can, for example, comprise a mechanical spring and/or be designed to be electrically, mechanically, pneumatically, or hydraulically actuatable.

The provision of a securing element advantageously enables a collision of the patient seat and/or of a patient positioned on the patient seat appropriately for the application with a housing portion of the magnetic resonance device to be avoided when the patient seat is transported in the direction of the patient receiving region. As a result, independent positioning of the patient on the patient seat can be enabled without the need for further work steps to check safety by the medical staff.

In a further aspect of the magnetic resonance device, according to the disclosure, the drive unit and the positioning unit are embodied to feed the patient seat along the spatial direction to the patient receiving region of the magnetic resonance device and simultaneously move the first portion variably relative to the second portion.

The movement of the patient seat by means of the drive unit and the relative positioning of the first portion and the second portion by means of the positioning unit are synchronized with one another. In particular, the movement of the patient seat by means of the drive unit and the relative positioning of the first portion and the second portion by means of the positioning unit can overlap, including in terms of time.

In one aspect, the magnetic resonance device has a control unit, which is embodied to actuate the drive unit and the positioning unit, feed the patient seat along the spatial direction to a patient receiving region of the magnetic resonance device, and simultaneously position the first portion relative to the second portion. The control unit can be designed as a stand-alone control unit or integrated into a control unit of the magnetic resonance device.

The control unit is preferably embodied to avoid a collision of the patient seat and/or of a patient positioned on the patient seat appropriately for the application with a housing portion of the magnetic resonance device.

In one aspect, the drive unit and the positioning unit are embodied to arrange a portion of the patient seat with a headrest and/or the head of a patient positioned on the patient seat appropriately for the application in an imaging volume of the magnetic resonance device.

In a further aspect, the drive unit and/or the positioning unit are embodied to automatically position a diagnostically relevant body region of a patient positioned on the patient seat appropriately for the application at least along a first spatial direction and a second spatial direction, which is aligned orthogonally to the first spatial direction, in an imaging volume of the magnetic resonance device.

A magnetic resonance device according to the disclosure enables a diagnostically relevant body region of a patient to be automatically positioned along a complex two-dimensional or three-dimensional movement trajectory in the imaging volume of the magnetic resonance device. As a result, it is possible to avoid a plurality of successive, one-dimensional movements of the patient seat when the patient is positioned in the image recording region and to reduce the time required for patient positioning.

In one aspect, the magnetic resonance device according to the disclosure comprises at least one sensor, which is embodied to ascertain information about a relative position of the patient seat and/or of a patient positioned on the patient seat appropriately for the application and a housing portion of the magnetic resonance device.

The sensor can be connected to a control unit of the magnetic resonance device by means of a signal connection. The control unit preferably has a computing unit, which is embodied to process data with information captured by the sensor about the relative position of the patient seat and/or the patient positioned on the patient seat appropriately for the application and the housing portion of the magnetic resonance device and to determine a positioning instruction in dependence on the data captured by the sensor. The positioning instruction can comprise movement information, in particular time-dependent movement information, which can be output by means of the control unit as control commands for the drive unit and/or the positioning unit. The positioning instruction preferably enables positioning of a diagnostically relevant body region of the patient in the imaging volume of the magnetic resonance device without causing a collision of the patient seat and/or the patient with a housing portion of the magnetic resonance device.

The sensor can, for example, be designed as an optical sensor, in particular, a distance sensor or a camera. The magnetic resonance device preferably comprises a plurality of sensors, which are embodied to ascertain the position of the patient seat and/or the patient positioned on the patient seat appropriately for the application relative to the housing portion of the magnetic resonance device. The computing unit can be embodied to apply a position determination method and/or an image recognition method for ascertaining a position of the patient seat and/or the patient arranged on the patient seat appropriately for the application relative to a housing portion of the magnetic resonance device in order to determine the positioning instruction.

In a further aspect, a computing unit of the magnetic resonance device is embodied to ascertain the positioning instruction in dependence on a patient model. The computing unit can be connected to a database, in particular a radiological information system (RIS), a local data storage, a cloud, or a comparable database containing patient information and/or a patient model.

In particular, the computing unit can be embodied to select and/or adapt a patient model in dependence on the data captured by the sensor and/or the patient information. The computing unit can furthermore be embodied to determine the positioning instruction in dependence on the selected and/or adapted patient model. The positioning instruction can furthermore be determined based on information about the dimensions of the patient seat and the magnetic resonance device, which can be provided by means of the database. It is, however, likewise conceivable that the positioning instruction is determined based on the patient model and the data captured by the sensor.

A housing portion of the magnetic resonance device can, for example, be part of an enclosure of the main magnet or the patient receiving region. In particular, a housing portion can be any part or portion of the magnetic resonance device that is arranged in the vicinity of a movement trajectory of the patient seat realized by means of the drive unit and/or the positioning unit and/or protrudes into a possible movement path of the patient seat.

A magnetic resonance device according to the disclosure with a sensor can advantageously avoid a collision between the patient seat and/or the patient and a housing portion. Ascertaining a positioning instruction and outputting corresponding control commands enables a diagnostically relevant body region to be positioned in a time-efficient and automatic manner in the imaging volume, whereby manual monitoring and/or patient positioning on the part of the medical staff can advantageously be dispensed with.

In a further aspect, the magnetic resonance device according to the disclosure has a sensor, which is embodied to determine information about a spatial arrangement of the at least one antenna element relative to a portion of the patient seat.

The sensor can be embodied to capture data containing information about the spatial arrangement of the at least one antenna element relative to the portion of the patient seat. The sensor can coincide with a sensor in an above-described aspect. For example, the sensor can be embodied as an optical sensor (for example, an infrared camera, a 2D camera, a 3D camera) or a distance sensor (for example, a laser distance sensor, a position encoder, or a Hall sensor). However, the sensor can also be designed as any mechanical sensor, which is embodied to ascertain a movement of the at least one antenna element relative to a portion of the patient seat. For example, the mechanical sensor can be embodied to be deformed as a result of the relative movement of the at least one antenna element and of the portion of the patient seat. It is, however, likewise conceivable that the sensor has a measuring path that varies in dependence on the relative movement of the at least one antenna element and of the portion of the patient seat. The sensor can furthermore have a measuring transducer, which converts information about the spatial arrangement of the at least one antenna element relative to the portion of the patient seat captured by the sensor into an electrical signal, preferably digital data.

The portion of the patient seat can be a first portion and/or a second portion, in particular, a seat surface, a backrest, and/or a headrest. In a preferred aspect, the portion is a headrest of the patient seat.

According to the disclosure, the drive unit and/or the positioning unit are embodied to position the diagnostically relevant body region of the patient in dependence on the information about the spatial arrangement of the at least one antenna element relative to the portion of the patient seat in the imaging volume.

The magnetic resonance device can comprise a computing unit, which is embodied to process captured data or electrical signals from the sensor. The computing unit is preferably embodied to ascertain a positioning instruction that can be used to position the diagnostically relevant body region of the patient in the imaging volume. The positioning instruction can comprise corresponding control commands for the drive unit and/or the positioning unit. It is likewise conceivable that the computing unit and/or a control unit are embodied to convert the positioning instruction into corresponding control commands. The control commands are preferably output to the drive unit and/or the positioning unit by means of a control unit. The computing unit and the control unit can be designed as a stand-alone unit or integrated into a control unit of the magnetic resonance device.

A magnetic resonance device according to the disclosure allows automatic detection of a diagnostically relevant body region and/or the dimensions of the diagnostically relevant body region of the patient in dependence on the arrangement of the at least one antenna element relative to the portion of the patient seat. As a result, it is possible to achieve particularly cost-effective and/or technically robust automated iso-centering (i.e., an application-appropriate arrangement of the diagnostically relevant body region in the imaging volume) of the diagnostically relevant body region.

In a further aspect the magnetic resonance device according to the disclosure has a locking mechanism with a first part and a second part. The first part and the second part of the locking mechanism are designed as complementary to one another and are embodied to mechanically engage with one another.

The first part and the second part of the locking mechanism can be connected to one another by means of a reversible mechanical connection, in particular, a force-fitting connection and/or a form-fitting connection. For example, the first part and the second part can be designed as complementary counterparts of a latching mechanism, a plug-in mechanism, a clamping mechanism, a suspension mechanism, or the like.

In a preferred aspect, the first part is funnel-shaped, while the second part is spherical or conical. It is conceivable that the funnel-shaped first part is embodied to receive the spherical second part and thereby center it in the direction of a center point of the funnel-shaped first part. Furthermore, the funnel-shaped first part can have a recess, in particular a bulge or a cylindrical cavity, at a narrowest cross section. The recess can be embodied to receive the spherical second part and prevent movement of the second part along two mutually orthogonally aligned spatial directions, in particular, the Y direction and the X direction of the magnetic resonance device. The funnel-shaped first part preferably enables limited rotation of the spherical first part, so that a headrest connected to the first part and/or a second portion of the patient seat connected to the first part can still be moved by means of the connecting element and/or the positioning unit.

In one aspect, the first part has a stop element, which is embodied to prevent movement of the second part along the Z-axis of the magnetic resonance device or the longitudinal axis of the patient receiving region. For example, the stop element can be designed as a wall within the recess of the funnel-shaped first part or as a conical wall of the funnel-shaped first part.

According to the disclosure, the second part is mechanically connected to the patient seat. For example, the second part can be attached to the first portion, the second portion, and/or the headrest. In a preferred aspect, the second part is attached to the headrest of the patient seat.

The first part is arranged within the patient receiving region and mechanically connected to a housing portion of the magnetic resonance device. The first part is preferably mechanically connected to a housing portion of the patient receiving region, in particular a holding structure for the patient receiving region and/or the magnetic holding structure.

The locking mechanism is embodied to limit or prevent movement of a portion of the patient seat along a spatial direction. The second part is preferably attached to a headrest of the patient seat, so that the locking mechanism is embodied to limit movement of the headrest in at least one spatial direction, in particular, a Z direction of the magnetic resonance device or a patient access direction.

It is conceivable that the locking mechanism is embodied to limit or prevent movement of the portion of the patient seat in at least two spatial directions aligned orthogonally to one another, in particular, a Z direction and a Y direction of the magnetic resonance device.

A locking mechanism according to the disclosure enables the patient seat to be fixed in the patient receiving region. Hence, a movement of a portion of the patient seat relative to the imaging volume of the magnetic resonance device during a magnetic resonance examination can advantageously be avoided. This can, in particular, be relevant for patient seats in which a relative arrangement of the portions can be changed manually by the patient by changing a posture of the patient (such as, for example, leaning back or leaning forward into an upright sitting position).

A locking mechanism according to the disclosure furthermore allows the use of unattached patient seats or patient seats with a lighter and/or less stable construction. The use of such patient seats advantageously enables the costs of the patient seat and/or the magnetic resonance device to be reduced.

Furthermore, the locking mechanism can be embodied to reduce or avoid oscillation and/or vibration of the patient seat, in particular the headrest of the patient seat, when the first part engages with the second part appropriately for the application. Oscillation and/or vibration of the patient seat can, for example, be the result of electromagnetic and/or mechanical forces based on gradient activity of the magnetic resonance device during the magnetic resonance examination and/or a movement of the patient on the patient seat.

A locking mechanism according to the disclosure can advantageously contribute to stabilizing a patient seat, in particular a headrest of the patient seat, against oscillations and/or vibrations during a magnetic resonance examination or can partially or completely prevent them.

In a further aspect, the locking mechanism of the magnetic resonance device according to the disclosure has a drive, which is embodied to affect an engagement of the first part and the second part in dependence on an activation signal when the patient seat is located in an application-appropriate position relative to the patient receiving region for performing the magnetic resonance measurement.

The locking mechanism can, for example, have an electric, hydraulic, or pneumatic drive. The drive is preferably embodied to bring the first part and the second part together so that the first part and the second part mechanically engage with one another or are mechanically connected to one another. The drive is preferably embodied to activate the locking mechanism from outside a volume enclosed by the main magnet. For this purpose, the drive can be positioned outside the patient receiving region or outside a main magnetic field of the magnetic resonance device. The drive preferably has a mechanical, hydraulic, or pneumatic positioning unit, which is embodied to bring together the first part and the second part of the locking mechanism as a result of activation by the drive.

In a preferred aspect, the drive is embodied to be actuated by means of an activation signal of a control unit of the magnetic resonance device. The control unit can, in particular, be embodied to activate the locking mechanism after the diagnostically relevant body region of the patient has been iso-centered.

In one example, the funnel-shaped first part has a positioning unit, which is embodied to move the first part relative to the second part in the direction of the second part as a result of actuation by means of the drive. In a further example, the spherical second part has a positioning unit, which is embodied to move the second part relative to the first part in the direction of the first part as a result of actuation by means of the drive. For example, the positioning unit can comprise a shaft, in particular a spindle, which can be deflected by means of the drive along a longitudinal direction of the patient receiving region or a Z direction of the magnetic resonance device.

A locking mechanism according to the disclosure enables a manual work step of locking the patient seat to be dispensed with. Furthermore, the locking mechanism according to the disclosure enables greater freedom in patient positioning, since the positioning unit can bridge a distance between the first part and the second part of the locking mechanism and hence the patient seat does not have to be transported into the patient receiving region until the second element rests against a stop element of the first element.

### DESCRIPTION OF THE DRAWINGS

Further advantages and details emerge from the following description of exemplary aspects in conjunction with the drawings. The drawings show schematically:

FIG. 1 illustrates a conventional magnetic resonance device,

FIG. 2 illustrates an aspect of a magnetic resonance device according to the disclosure,

FIG. 3 illustrates an aspect of a magnetic resonance device according to the disclosure,

FIGS. 4a and 4b illustrate an aspect of a patient seat according to the disclosure,

FIG. 5 illustrates an aspect of a patient seat according to the disclosure,

FIG. 6 illustrates an aspect of a patient seat according to the disclosure,

FIG. 7 illustrates an aspect of a patient seat according to the disclosure, and

FIG. 8 illustrates an aspect of a magnetic resonance device according to the disclosure.

### DETAILED DESCRIPTION

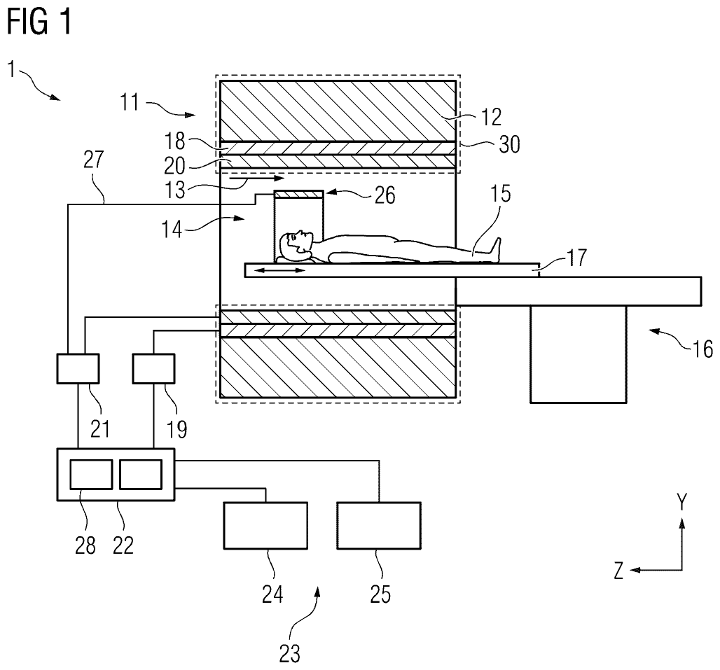

FIG. 1 shows a conventional magnetic resonance device 1. The magnetic resonance device 1 comprises a field generation unit 11, which has a main magnet 12 with one or more permanent magnets, electromagnets, or superconducting magnets for generating a strong and, in particular, homogeneous main magnetic field 13 (BO magnetic field). In addition, the magnetic resonance device 1 comprises a patient receiving region 14 for receiving a patient 15. In the exemplary aspect shown, the patient receiving region 14 is cylindrical in shape and is enclosed by the main magnet 12 in a circumferential direction. However, in principle, aspects of the patient receiving region 14 that deviate from this example are also conceivable. The patient receiving region 14 can substantially coincide with an image recording region of the magnetic resonance device 1.

In the example shown in FIG. 1, the patient 15 can be positioned by means of a patient support apparatus 16 of the magnetic resonance device 1 in the patient receiving region 14. The patient support apparatus 16 has a horizontally movable patient table 17 for this purpose.

The field generation unit 11 has a gradient system with at least one gradient coil 18 for generating a magnetic gradient field, which is used for spatial encoding during a magnetic resonance examination. The gradient coil 18 is actuated by means of a gradient control unit 19 of the magnetic resonance device 1. It is conceivable that the gradient system comprises a plurality of gradient coils 18 for generating magnetic gradient fields along different spatial directions, preferably aligned orthogonally to one another.

The field generation unit 11 also comprises a radio-frequency system with a radio-frequency coil, which, in the present exemplary aspect, is embodied as a body coil 20 that is permanently integrated into the magnetic resonance device 1. The body coil 20 is designed to excite nuclear spins that are located in the main magnetic field 13 generated by the main magnet 12. The body coil 20 is actuated by a radio-frequency control unit 21 of the magnetic resonance device 1 and emits radio-frequency excitation pulses into the image recording region, which is substantially formed by the patient receiving region 14 of the magnetic resonance device 1. The body coil 20 can furthermore be embodied to receive magnetic resonance signals and form a receiving unit or part of a receiving unit of the magnetic resonance device 1.

To control the magnetic resonance device 1, in particular the gradient control unit 19 and the radio-frequency control unit 21, the magnetic resonance device 1 has a control unit 22. The control unit 22 is, in particular, embodied to coordinate the performance of an imaging sequence, such as, for example, a GRE (gradient echo) sequence, a TSE (turbo spin echo) sequence, or a UTE (ultra-short echo time) sequence. In addition, the control unit 22 comprises a computing unit 28 for evaluating magnetic resonance signals captured during a magnetic resonance examination with an imaging sequence.

The magnetic resonance device 1 can comprise a user interface 23, which has a signal connection with the control unit 22. Control information, such as, for example, imaging parameters of the magnetic resonance examination, can be displayed on a display unit 24, for example, on at least one monitor, of the user interface 23. The display unit 24 can, in particular, be designed to provide a graphical user interface with the representation of a relevant body region of the patient 15. Furthermore, the user interface 23 has an input unit 25 by means of which parameters of a magnetic resonance measurement can be entered or changed by a user.

The magnetic resonance device 1 can have further components, such as, for example, a local coil 26. The local coil 26 can be positioned in an application-appropriate position on a diagnostically or therapeutically relevant body region of the patient 15. The local coil 26 preferably has a plurality of antenna elements, which are embodied to capture magnetic resonance signals from the relevant body region of the patient 15 and transmit them to the computing unit 28 and/or the control unit 22. For this purpose, the local coil can be connected to the radio-frequency control unit 21 and the control unit 22 by means of an electrical connection line 27 or another signal connection. Similar to the body coil 20, the local coil 26 can also be embodied to excite nuclear spins in the jaw region 31 of the patient 15. For this purpose, the local coil 26 can be actuated by the radio-frequency control unit 21.

The field generation unit 11 and a magnetic holding structure are usually enclosed in a housing 30. The housing 30 can be embodied to shield components of the magnetic resonance device 1 from external influences and/or to ensure the patient 15 is protected from contact.

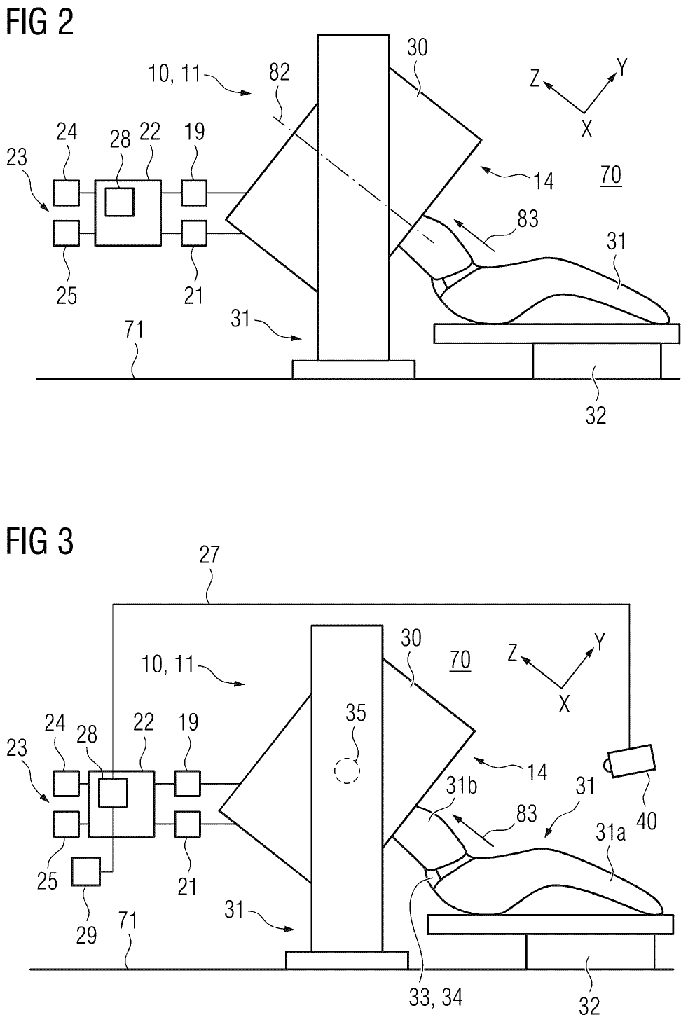

FIG. 2 shows an aspect of a magnetic resonance device according to the disclosure. In principle, the functions and components of the magnetic resonance device 10 depicted in FIG. 2 correspond to the above-described functions and components of a conventional magnetic resonance device 1 (see FIG. 1).

For example, the magnetic resonance device 10 can be embodied to perform a magnetic resonance examination of a head region, a jaw region and/or a dental region of a patient 15. However, the magnetic resonance device according to the disclosure 10 can likewise be embodied to perform cardiac imaging, neurological imaging, urological imaging, orthopedic imaging, prostate imaging, or imaging of other body regions of the patient 15, in particular extremities.

In the aspect shown in FIG. 2, a longitudinal axis 82 of the patient receiving region 14 (or a longitudinal axis 82 of the magnetic resonance device 10) is arranged inclined relative to a horizontal 71, in particular, a horizontally aligned floor surface 71 of an examination room 70. In the present example, the patient access direction 83 coincides with the Z direction of the magnetic resonance device 10 and is also inclined relative to the floor surface 71. The inclination of the magnetic resonance device 10 enables a magnetic resonance examination of a seated patient and, hence, a reduction in the space required by the magnetic resonance device 10 with the patient seat 31.

In the present case, the patient seat 31 has a drive unit 32, which is embodied to move the patient seat 31 variably along a spatial direction. In the example shown, the drive unit is embodied to move the patient seat 31 parallel to the floor surface 71. It is, however, likewise conceivable that the drive unit is embodied to move the patient seat 31 at an angle to the floor surface 71, in particular along the Z direction of the magnetic resonance device 10. The drive unit can also be embodied to move the patient seat 31 variably along a plurality of spatial directions, in particular spatial directions that are aligned orthogonally to one another.

FIG. 3 shows an aspect of the magnetic resonance device according to the disclosure 10 with a sensor 40. In the present case, the sensor 40 is designed as a camera embodied to capture image data in the examination room 70 with the magnetic resonance device 10 and transmit it by means of the signal connection 27 to the computing unit 28 of the magnetic resonance device 10.

The computing unit 28 is embodied to ascertain a relative position of the patient seat 31 and/or a relative position of a patient positioned on the patient seat appropriately for the application and a housing portion of the housing 30 of the magnetic resonance device 10 in dependence on the image data captured by the sensor 40. The computing unit 28 is furthermore embodied to determine a positioning instruction in dependence on image data of the sensor 40. The positioning instruction comprises time-dependent movement information, which is output as control commands to the drive unit 32 and/or the positioning unit 33 of the patient seat 31 by means of the control unit 22. The control unit 22 can accordingly be embodied to position a diagnostically relevant body region of the patient in an imaging volume of the magnetic resonance device 10 by means of the drive unit 32 and/or the positioning unit 33 in dependence on the positioning instruction. In a preferred aspect, the control unit 22 is embodied to feed the patient seat 31 along the patient access direction 83 to the patient receiving region 14 by means of actuating the drive unit 32 and the positioning unit 33 of the magnetic resonance device 10 and to simultaneously position the second portion 31b relative to the first portion 31a.

It is likewise conceivable that the sensor 40 is embodied to capture information about a spatial arrangement of an antenna element 50 (see FIG. 5) relative to a portion of the patient seat 31. In the case of a camera as a sensor 40, the computing unit 28 can be embodied to determine the spatial arrangement of the antenna element 50 relative to a portion of the patient seat 31 in dependence on the image data captured by the sensor 40. The computing unit 28 can furthermore be embodied to ascertain a positioning instruction that can be used to position the diagnostically relevant body region of the patient in the imaging volume 35 of the magnetic resonance device 10. As described above, the control unit 22 can be embodied to output corresponding control commands to the drive unit 32 and/or the positioning unit 33 in dependence on the positioning instruction.

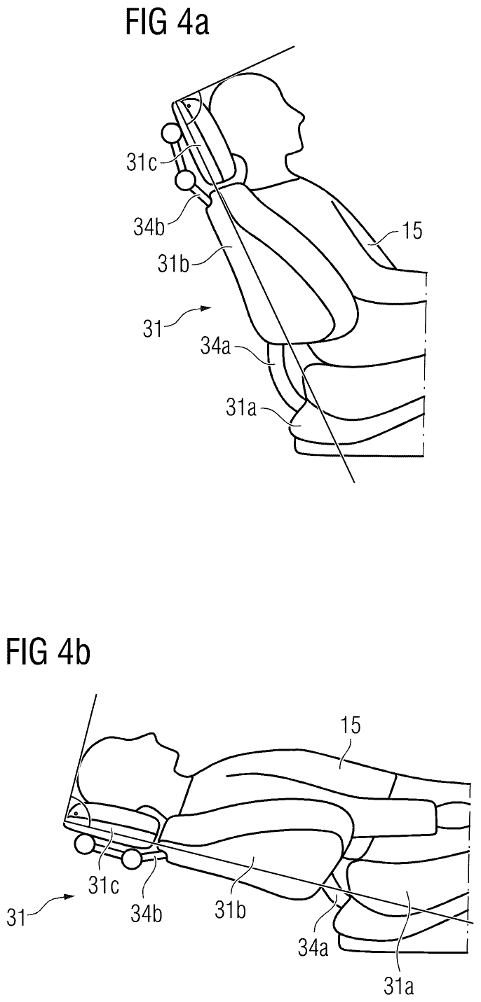

FIG. 4a and FIG. 4b show an aspect of the patient seat 31 according to the disclosure with a seat surface 31a, a backrest 31b, and a headrest 31c. The seat surface 31a and the backrest 31b are connected by means of a connecting element 34a, while the backrest 31b and the headrest 31c are connected by means of a connecting element 34b. A patient 15 is positioned on the patient seat 31 appropriately for the application.

The connecting elements 34a and 34b can be embodied to enable a variable relative movement between the portions 31a, 31b, and 31c of the patient seat 31. In the example shown, the connecting element 34a is embodied to enable a movement of the backrest 31b relative to the seat surface 31a. On the other hand, the connecting element 34b enables a movement of the headrest 31c relative to the backrest 31b. In the present case, the connecting element 34a comprises a joint, which enables tilting of the backrest 31b relative to the seat surface 31a along a predetermined movement trajectory. The connecting element 34b comprises a plurality of joints, which enable positioning and tilting of the headrest 31c relative to the backrest 31b.

In the example shown in FIG. 4a, the patient 15 is substantially in an upright sitting position. It is conceivable that the longitudinal axis 82 of the patient receiving region 14 is inclined such that a magnetic resonance examination of the patient 15 can be performed in the upright sitting position. The patient seat 31 is preferably embodied to avoid an arrangement of the patient's head 15 relative to the headrest 31c when the patient 15 is leaning back on the patient seat 31, as shown in FIG. 4b. As a result, steps for preparing the patient 15, such as, for example, an arrangement of local coils 26 or antenna elements 50, can already be carried out on a patient 15 sitting upright, whereby the workload for medical staff can be reduced.

In a preferred aspect, the connecting elements 34a and 34b are embodied to move the headrest 31c and the backrest 31b variably relative to the seat surface 31a such that a position of the patient's head 15 relative to the headrest 31c remains substantially unchanged when the patient 15 is leaning back. As shown in FIG. 4a and FIG. 4b, an angle between the headrest 31 and a tangent to the highest point of the patient's head 15 remains substantially unchanged when the patient 15 leans back on the patient seat 31.

In all aspects described herein, the patient seat 31 according to the disclosure can have passive or purely mechanical connecting elements 34, which enable a relative movement between the portions 31a, 31b, and/or 31c (31a-c) by interaction of a patient 15 or a member of the medical staff. Likewise, the connecting elements 34 can be coupled to active positioning units 33, which have a drive and allow an automated relative movement of the portions 31a, 31b, and/or 31c.

FIG. 5 shows an aspect of the patient seat 31 according to the disclosure with a guide element 53, which is embodied to move the antenna element 50 of the radio-frequency unit 51 variably relative to the headrest 31c.

In this example, the guide element 53 and the antenna element 50 are mechanically connected to the headrest 31c of the patient seat 31. The guide element 53 is embodied as a hinge, which enables the antenna element 50 to pivot or rotate about an axis defined by the guide element 53 (for example, an axis parallel to a sagittal plane and/or a frontal plane of the upper body of the patient 15). Hence, the antenna element 50 can be placed on the patient's head 15 from the side or be positioned at a predetermined distance to the patient's head 15.

In a preferred aspect, the patient seat 31 has at least two antenna elements 50 (not shown), which are arranged on opposite sides of the headrest 31c and flank the head of the patient 15 positioned on the patient seat 31 appropriately for the application from opposite sides.

It is conceivable that the guide element 53 and/or the headrest 31c have a bearing, which is embodied to move the antenna element 50 relatively along a parallel to the sagittal plane and/or the frontal plane, in particular, along a parallel to an intersecting line 80 of the sagittal plane with the frontal plane, of the upper body of the patient 15. As a result, as shown in FIG. 5, in addition to a dental region and/or jaw region of the patient 16, the antenna element 50 can also be arranged on other regions of the patient's head 15.

It is conceivable that a guide element 53 with an antenna element 50 is alternatively or additionally arranged on the backrest 31b and/or the seat surface 31a in order to enable a magnetic resonance examination on further or other body regions of the patient 15.

In the aspect of the patient seat 31 shown in FIG. 5, the headrest 31c, the backrest 31b, and the seat surface 31a can be moved variably relative to one another by a patient 15 or a member of the medical staff by means of the connecting elements 34a and 34b. It is, however, likewise conceivable that one or more connecting elements 34 are designed as positioning units 33 or have a positioning unit 33. As a result, a variable relative movement of the headrest 31c, the backrest 31b, and/or the seat surface 31a can take place in an automated manner. The positioning units 33a and/or 33b shown in FIG. 5 can, for example, be actuated or activated by means of drives integrated into the patient seat 31 as the result of actuation by the control unit 22.

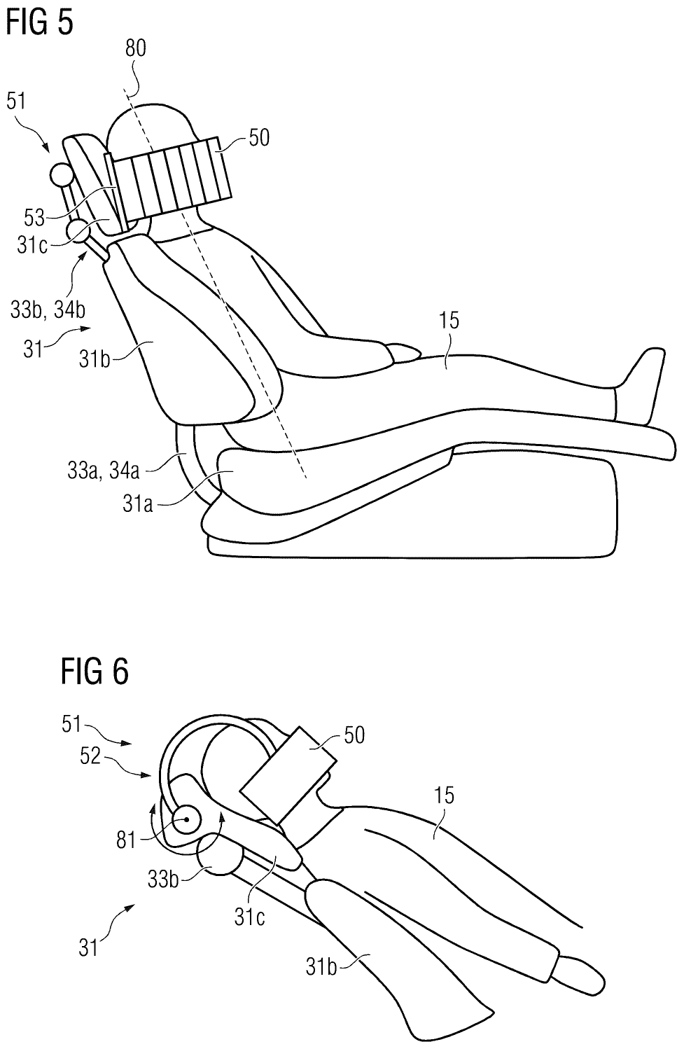

FIG. 6 shows an aspect in which the radio-frequency unit 51 of the patient seat 31 according to the disclosure has a pivoting mechanism 52. The pivoting mechanism 52 is embodied to pivot or rotate the antenna element 50 about an axis 81 that is aligned substantially parallel to a medial direction of the patient 15. The antenna element 50 can be connected to the pivoting mechanism 52 by means of a guide element, in particular a joint (not shown) in order to enable adaptation of a spatial position and/or alignment of the antenna element 50 relative to the pivoting mechanism 52.

The pivoting mechanism 52 is preferably integrated into the headrest 31c of the patient seat 31. It is, however, likewise conceivable that the pivoting mechanism 52 is integrated into other portions of the patient seat 31.

The radio-frequency unit 51 can, of course, comprise a plurality of antenna elements 50. The radio-frequency unit 51 preferably has two antenna elements 50, which are held by the pivoting mechanism 52 on opposite sides of the headrest 31c and flank the head of the patient 15 positioned in the patient seat 31 appropriately for the application from opposite sides.

The radio-frequency unit 51 of the patient seat 31 is preferably part of the radio-frequency system of the magnetic resonance device 10. For example, one or more antenna elements 50 of the patient seat 31 can be connected to the radio-frequency control unit 21 of the magnetic resonance device 10 (see FIG. 1 to FIG. 3) by means of a signal connection and actuated thereby. It is, in particular, conceivable that the radio-frequency unit 51 is connected to the radio-frequency control unit 21 by means of an electrical connection line integrated into the patient seat 31 in order to enable actuation of the antenna elements 50. Integration of the antenna elements 50 into the patient seat 31 advantageously enables electrical lines located in a free space of the patient receiving region 14 to be avoided.

A radio-frequency unit 51 with an antenna element 50 according to FIG. 5 or FIG. 6 enables the antenna element 50 to be arranged in an application-appropriate position on the diagnostically relevant body region by the patient 15. Even if the antenna element 50 is arranged on the patient 15 by a member of the medical staff, the antenna element 50 only has to be folded or pivoted into the application-specific position before the patient 15 is transported by means of the patient seat 31 into the patient receiving region 14 in the application-appropriate position for performing the magnetic resonance examination.

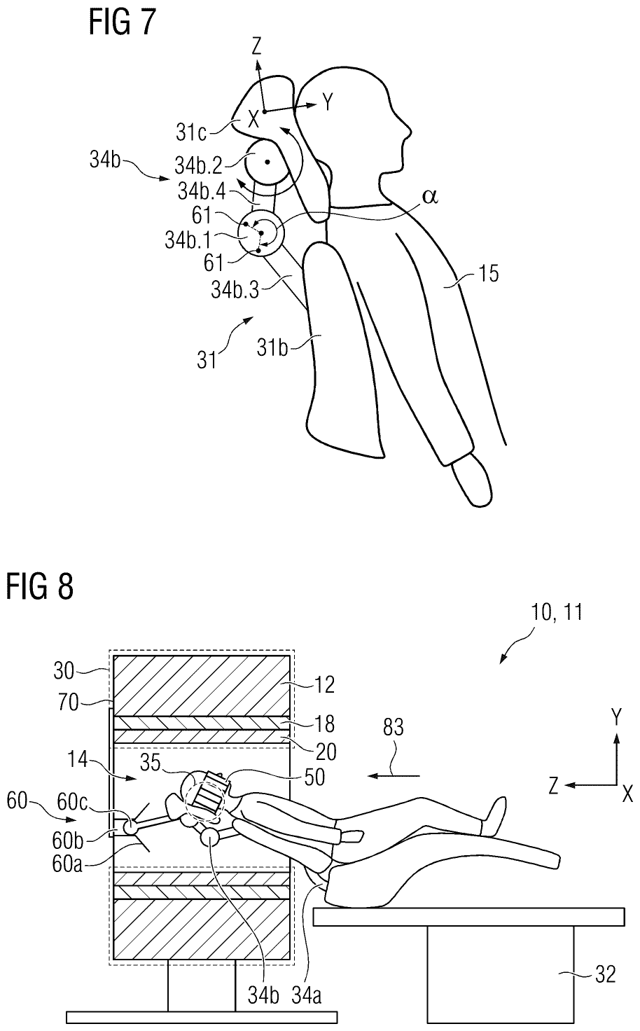

FIG. 7 shows an aspect of a patient seat 31 according to the disclosure with a connecting element 34b. In the present case, the connecting element 34b is embodied to move the headrest 31c relative to the backrest 31b of the patient seat. The connecting element 34b, in particular, has two bearings or joints 34b.1 and 34b.2, which enable positioning and alignment of the headrest 31c relative to the backrest 31b.

For example, the joint 34b.2 is embodied to enable rotation of the headrest 31c about an axis of rotation defined by the joint 34b.2. The axis of rotation defined by the joint 34b. 2 is preferably aligned substantially parallel to a medial direction of the patient 15, in particular an X direction of the patient seat 31 and/or the magnetic resonance device 10 (see FIG. 3). It is, however, likewise conceivable that the joint 34b.2 is designed as a ball joint, which enables a substantially three-dimensional alignment of the headrest 31c relative to the backrest 31b.

The joint 34b.1 is preferably embodied to enable a change in an angle between two struts 34b.3 and 34b. 4, which connect the headrest 31c to the backrest 31b.

In a preferred aspect, the joints 34b.1 and/or 34b.2 have mechanical resistances, which only allow a movement of the headrest 31c relative to the backrest 31b when a predetermined force is exceeded. As a result, the patient seat 15 can be held in a desired configuration permanently or for the duration of the magnetic resonance examination depending on a posture of the patient 15, in particular a weight force exerted by the body of the patient 15 on the portions 31c and 31b. The desired configuration can, in particular, be characterized by the fact that a diagnostically relevant body region of the patient 15, such as, for example, a portion of the brain, a portion of a jaw region, and/or a portion of a dental arch, are positioned in the imaging volume 35 of the magnetic resonance device 10 (see FIG. 3 and FIG. 8). It is conceivable that the connecting element 34b has means for adjusting the mechanical resistances (not shown) of the joints 34b.1 and/or 34b.2. Such means can, for example, comprise a screw mechanism, which enables the adaptation of a friction force between moving parts of the joints 34b.1 and/or 34b.2.

The connecting element 34b can furthermore comprise a positioning unit 33 (not shown) or be embodied as part of a positioning unit 33. The positioning unit 33 can, for example, comprise a drive, which is embodied to move the joints 34b.1 and/or 34.b2 appropriately for the application. It is conceivable that the joints 34b.1 and/or 34b.2 have elements, which are embodied to be driven by means of a motor integrated into the patient seat 31 or an external motor. However, the positioning unit 33 can also be embodied to move the struts 34b. 3 and 34b. 4 relative to one another by means of tie rods, pistons, or the like. One or more positioning units 33 can be used to transfer the patient seat 31 into a desired configuration in a partially or completely automated manner.

Regardless of the presence of a positioning unit 33, the patient seat 31 according to the disclosure can be embodied to move a body region, in particular the head, of the patient 15 along an anterior-posterior direction and a superior-inferior direction of the upper body of the patient 15. In the example shown in FIG. 7, the anterior-posterior direction corresponds to the Y direction. On the other hand, the superior-inferior direction corresponds to the Z direction. However, it is furthermore conceivable that the headrest 31c, and also the backrest 31b, can be pivoted about an axis aligned parallel to the X-direction by means of a correspondingly designed connecting element 34 and/or a positioning unit 33.

The example of the patient seat 31 shown in FIG. 7 has a securing element 61, which is embodied to limit an extent of an alignment of the headrest 31c relative to the backrest 31b in order to avoid a collision of the patient seat 31 and/or of a patient 15 positioned on the patient seat 31 appropriately for the application with a housing portion of a housing 30 of a magnetic resonance device 10. For example, the securing element 61 can be embodied to limit excessive leaning back on the part of the patient 15 on the patient seat 31 in order to avoid a collision of the patient seat 31 with a housing 30, as shown in FIG. 8.

In the present case, the securing element 61 has a plurality of stop elements, in particular pins or bolts. The stop elements are embodied to limit an angle α between the two struts 34b.4 and 34b. 3 that can be adjusted by means of the joint 34b. 1. For example, the stop elements can form fixed stop points for the struts 34b.4 and 34b.3 or limit an opening angle α of the joint 34b. 1.

Of course, the joint 34b.2 or a connecting element 34b.3 (not shown), which mechanically connects the seat surface 31a to the backrest 31b, can also have a securing element 61.

FIG. 8 shows an aspect of a magnetic resonance device according to the disclosure 10 with a locking mechanism 60.

The locking mechanism 60 has a funnel or cone 60a with a cylindrical recess 60b and a coupling element 60c with a spherical end. The cone 60a and the coupling element 60c are designed as complementary to one another and embodied to mechanically engage with one another. In the present case, the coupling element 60c is mechanically connected to the patient seat 31, in particular the headrest or the connecting element 34b. The cone 60a is arranged within the patient receiving region 14 and mechanically connected to a magnetic holding structure 70 of the magnetic resonance device 10.

The locking mechanism 60 is embodied to limit a movement of a portion of the patient seat 31 along a spatial direction. For example, the locking mechanism 60 prevents a movement of the patient seat 31 in the direction of the end of the patient receiving region 14 with the locking mechanism 60 when the coupling element 60c abuts the wall in the cylindrical recess 60b. Furthermore, a freedom of movement of the spherical end of the coupling element 60c along the Y direction can be limited by the lateral surface of the cylindrical recess 60b. However, the coupling element 60b can be attached to a portion of the patient seat 31 with a joint so that restricted positioning of the headrest and/or the backrest of the patient seat along the Y direction is enabled by means of the connecting elements 34b and/or 34a. Furthermore, the spherical end of the coupling element 60c can be supported rotatably in the cylindrical recess 60b in order to enable restricted positioning of the headrest and/or the backrest of the patient seat along the Y direction. Herein, the rotation of the spherical end of the coupling elements 60c can be limited by an opening angle of the cone 60a.

In the aspect shown in FIG. 8, the locking mechanism 60 is designed as passive. Therefore, engagement of the coupling element 60c in the cone 60a and/or the cylindrical recess 60b is substantially realized by a movement of the patient seat 31 along the Z direction by means of the drive unit 31 and/or a positioning unit 33.

It is, however, likewise conceivable that the cone 60a is coupled to the cylindrical recess 60b and/or the coupling element 60c is coupled to a drive, which is embodied to move the two parts of the locking mechanism 60 toward one another. For example, the coupling element 60c can have a spindle-shaped shaft that enables the spherical end to be positioned along the Z direction by means of a gear. In a further example, the patient seat 31 can have a hydraulic or pneumatic drive, for example a piston. Such a drive can be designed to deflect the coupling element 60c along the Z direction. It is, however, likewise conceivable that the cone 60a can be positioned along the Z direction and/or the Y direction by means of a suitable drive.

FIG. 4 to FIG. 8 show a patient 15 positioned on a patient seat 31 according to the disclosure appropriate for the application. However, the examples described here should not be understood to be restrictive with respect to an application-appropriate position of the patient 15. For example, the position and/or posture of the patient 15 can deviate from the examples shown in dependence on the construction and/or design of the patient seat 31, while still being regarded as an application-appropriate position of the patient 15 on the patient seat 31.

Although the disclosure has been illustrated and described in greater detail by the preferred exemplary aspects, the disclosure is not restricted by the disclosed examples, and other variations can be derived herefrom by the person skilled in the art without departing from the scope of protection of the disclosure. In particular, features of individual aspects can be combined with features of other aspects provided that such a combination has not been explicitly excluded in the description.

1. A patient seat for supporting a patient during a magnetic resonance examination, comprising:
a first portion;
a second portion;
a connecting element;
a radio-frequency unit with at least one antenna element; and
a drive unit,
wherein the first portion and the second portion form parts of a receiving surface for the patient,
wherein the connecting element mechanically connects the first portion to the second portion and is configured to enable a variable relative movement between the first portion and the second portion,
wherein the at least one antenna element of the radio-frequency unit is configured to receive signals in a power and frequency range of a magnetic resonance examination, and
wherein the drive unit is configured to move the patient seat variably along a spatial direction.

2. The patient seat as claimed in claim 1, further comprising:
a positioning unit, which is configured to automatically position the first portion variably relative to the second portion.

3. The patient seat as claimed in claim 1, wherein the drive unit is configured to position the patient seat variably along a first spatial direction and a second spatial direction, which is aligned orthogonally to the first spatial direction.

4. The patient seat as claimed in claim 1, further comprising:
a headrest; and
a further connecting element,
wherein the further connecting element is configured to position the headrest variably relative to the first portion and/or the second portion, and wherein the radio-frequency unit is arranged on the headrest.

5. The patient seat as claimed in claim 4, wherein the further connecting element is configured to enable variable positioning of the headrest substantially parallel to an anterior-posterior direction and/or a superior-inferior direction of an upper body of a patient positioned on the patient seat appropriately for an application.

6. The patient seat as claimed in claim 1, wherein the connecting element is configured to move the first portion variably relative to the second portion such that a relative position of a head of a patient positioned on the patient seat appropriately for an application and a portion of the patient seat remains substantially unchanged.

7. The patient seat as claimed in claim 1, wherein the radio-frequency unit has a guide element, and wherein the guide element is configured to move the at least one antenna element variably relative to a portion of the patient seat.

8. The patient seat as claimed in claim 1, wherein the radio-frequency unit has a pivoting mechanism, which is configured to pivot the at least one antenna element about an axis aligned substantially parallel to a medial direction of the patient when the patient is positioned on the patient seat appropriately for an application.

9. A magnetic resonance device for performing a magnetic resonance examination on a patient arranged in a patient receiving region of the magnetic resonance device, comprising:
a patient seat as claimed in claim 1,
wherein the drive unit is configured to move the patient seat variably relative to the patient receiving region of the magnetic resonance device at least along a spatial direction.

10. The magnetic resonance device as claimed in claim 9, further comprising:
a securing element, which is configured to limit an extent of an alignment of the first portion relative to the second portion in order to avoid a collision of the patient seat and/or a patient positioned on the patient seat appropriately for an application with a housing portion of the magnetic resonance device.

11. The magnetic resonance device as claimed in claim 9 with a patient seat for supporting a patient during a magnetic resonance examination, comprising:
a first portion;
a second portion;
a connecting element;
a radio-frequency unit with at least one antenna element;
a drive unit; and
a positioning unit, which is configured to automatically position the first portion variably relative to the second portion,
wherein the first portion and the second portion form parts of a receiving surface for the patient,
wherein the connecting element mechanically connects the first portion to the second portion and is configured to enable a variable relative movement between the first portion and the second portion,
wherein the at least one antenna element of the radio-frequency unit is configured to receive signals in a power and frequency range of a magnetic resonance examination,
wherein the drive unit is configured to move the patient seat variably along a spatial direction, and
wherein the drive unit and the positioning unit are configured to feed the patient seat along the spatial direction to the patient receiving region of the magnetic resonance device and to simultaneously move the first portion variably relative to the second portion.

12. The magnetic resonance device as claimed in claim 11, wherein the drive unit and/or the positioning unit are configured to automatically position a diagnostically relevant body region of a patient positioned on the patient seat appropriately for an application at least along a first spatial direction and a second spatial direction, which is aligned orthogonally to the first spatial direction in an imaging volume of the magnetic resonance device.

13. The magnetic resonance device as claimed in claim 12, further comprising:
a sensor, which is configured to determine information about a spatial arrangement of the at least one antenna element relative to a portion of the patient seat,
wherein the drive unit and/or the positioning unit of the patient seat are configured to position the diagnostically relevant body region of the patient in the imaging volume in dependence on the information about the spatial arrangement of the at least one antenna element relative to the portion.

14. The magnetic resonance device as claimed in claim 9, further comprising:
a locking mechanism with a first part and a second part,
wherein the first part and the second part are designed to be complementary to one another and are configured to mechanically engage with one another,
wherein the second part is mechanically connected to the patient seat,
wherein the first part is arranged within the patient receiving region and mechanically connected to a housing portion of the magnetic resonance device, and
wherein the locking mechanism is configured to limit a movement of a portion of the patient seat along a spatial direction.

15. The magnetic resonance device as claimed in claim 14, wherein the locking mechanism has a drive, which is configured to affect an engagement of the first part and the second part in dependence on an activation signal when the patient seat is located in an application-appropriate position relative to the patient receiving region for performing the magnetic resonance examination.
